(19) United States
(12) Patent Application Publication
(10) Pub. No.: US 2008/0287467 A1
(43) Pub. Date: Nov. 20, 2008

(54) Therapeutic Agent for Respiratory Diseases

(76) Inventors: Gen Tamura, Miyagi (JP); Masayuki Nara, Miyagi (JP); Miyuki Nagaoka, Shizuoka (JP)

Correspondence Address: SUGHRUE-265550, 2100 PENNSYLVANIA AVE. NW, WASHINGTON, DC, 20037-3213, US

(21) Appl. No.: 11/579,681

(22) PCT Filed: Apr. 28, 2005
(86) PCT No.: PCT/JP2005/008553

§ 371 Date: Nov. 6, 2006

(30) Foreign Application Priority Data
May 6, 2004 (JP) 2004-137563

(51) Int. Cl. A61K 31/52; A61P 11/06; A61P 11/08

(52) U.S. Cl. 514/263.1

(57) ABSTRACT

The invention provides a therapeutic agent for respiratory diseases, which is based on a new activity mechanism of the antagonism to the P2X4 receptor and by which fewer adverse activities of the existing β-stimulants on the cardiovascular system can be expected. The therapeutic agent of the invention is antagonistic to the P2X4 receptor present in bronchial smooth muscle and is used for the treatment of respiratory diseases caused by bronchocontraction such as asthma.

### TECHNICAL FIELD

The present invention relates to a therapeutic agent for respiratory diseases. More specifically, the invention relates to a therapeutic agent for respiratory diseases, which comprises a compound antagonistic to P2X receptor.

### BACKGROUND OF THE INVENTION

Bronchial asthma is a disease caused by the obstruction of airway due to airway contraction and inflammation, involving paroxysmal cough, wheezing and dyspnoea. Currently, β stimulants are used as bronchodilator. However, adverse effects on the cardiovascular system are problematic. Therefore, a bronchodilator based on a novel activity mechanism has been desired in addition to existing therapeutic agents for asthma.

Purine receptors are classified into P1 receptors and P2 receptors. The P1 receptors employ adenosine as the ligand, while the P2 receptors employ mainly adenosine-5′-triphosphate (ATP) and adenosine-5′-diphosphate (ADP) as the ligand. The P2 receptors are divided into P2 receptors of ion channel type in which the receptor proteins themselves constitute an ion channel (P2X receptor), and P2 receptors of metabolism-modulating type in which the receptors function by activating the G protein (P2Y receptor). The P2X receptors and the P2Y receptors are further classified into several subtypes, respectively.

Among the subtypes of the P2X receptors, it is known that P2X4 receptor is distributed in many tissues such as the central nervous system and is also localized in the airway smooth muscle (for example, “Cell Tissue Res., Germany, 2003, Vol. 313, pp. 159-165”). It is known that the P2X4 receptor is involved in pain, in particular (for example, “Nature, USA, 2003, Vol. 424, pp. 778-783”). However, these references never tell anything about the function of the P2X4 receptor in the airway smooth muscle.

By contrast, it is known that ATP contracts the airway smooth muscle (“Am. J. of Physiol-Lung Cell. Mol. Physiol., USA, 2002, Vol. 283, pp. 1271-1279”). When an ATP receptor antagonist can be created, such an ATP receptor antagonist can be used for therapeutically treating respiratory diseases caused by the contraction of the airway smooth muscle such as asthma. In “Am. J. of Physiol-Lung Cell. Mol. Physiol., USA, 2002, Vol. 283, pp. 1271-1279”, it is concluded that the contraction is induced via P2Y2 receptor or P2Y4 receptor.

The pamphlet of the International Publication 2002/71062 describes the efficacy in the prophylaxis or therapeutic treatment of hyperactive immune response for which the P2 receptor is responsible. However, the patent specification simply describes the therapeutic treatment of asthma by the regulation of immune reaction, namely the suppression of inflammation, and it does not disclose or suggest about the relation of the P2X receptor with the contraction of the airway smooth muscle.

### DISCLOSURE OF THE INVENTION

It is an object of the invention to provide a therapeutic agent for respiratory diseases for dilating bronchial tract on the basis of a novel activity mechanism, particularly a therapeutic agent for asthma.

In view of the problems described above, the inventors made investigations about the ATP activity in bronchial tract from various aspects. Consequently, the inventors first found that the contraction of the airway smooth muscle with ATP is a reaction via the P2X4 receptor. In other words, antagonists to the P2X4 receptor on the airway smooth muscle are useful for therapeutically treating respiratory diseases induced by the bronchocontraction such as asthma and can be used as a therapeutic agent for respiratory diseases. The therapeutic agent for respiratory diseases according to the invention is based on a novel activity mechanism which is not conventionally known and is expected that the adverse effects of the existing β stimulants on the cardiovascular system will be reduced.

Specifically, the invention relates to the followings.

1. A therapeutic agent for respiratory diseases, comprising a compound antagonistic to a P2X receptor.
2. The therapeutic agent for respiratory diseases according to 1 above, wherein the compound antagonistic to a P2X receptor is a compound satisfying the following conditions:

(1) the compound suppressing an inward current induced by ATP in the measurement with the patch cramp method;

(2) the compound antagonistic to a receptor which is not antagonized with pyridoxal phosphate-6-azophenyl-2′,4′-disulfonic acid and/or suramin; and

(3) the compound suppressing an inward current enhanced by ivermectin in the measurement with the patch cramp method.

3. The therapeutic agent for respiratory diseases according to 1 above, wherein the compound antagonistic to a P2X receptor has a purine skeleton.
4. The therapeutic agent for respiratory diseases according to 1 above, wherein the respiratory disease is asthma.
5. The therapeutic agent for respiratory diseases according to 1 above, which dilates a bronchus.
6. The therapeutic agent for respiratory diseases according to 1 above, wherein the therapeutic agent is abronchodilator.
7. The therapeutic agent for respiratory diseases according to 4 above, wherein the P2X receptor is a P2X4 receptor.
8. The therapeutic agent for respiratory diseases according to 5 above, wherein the P2X receptor is a P2X4 receptor.

The therapeutic agent for respiratory diseases according to the invention is antagonistic to the P2X4 receptor in the airway smooth muscle to suppress the contraction of the airway smooth muscle. Accordingly, the therapeutic agent is useful for the prevention and/or treatment of respiratory diseases such as asthma and chronic obstructive lung disease.

According to the invention, bronchodilation means the dilation of bronchial smooth muscle contracted due to the involvement of the P2X receptor.

The therapeutic agent for respiratory diseases according to the invention may satisfactorily have additional activities such as anti-inflammatory and inhibition of mucous secretion, in addition to the effects of inhibition of the contraction of bronchial smooth muscle.

In the present specification, the P2X receptor means an ion channel composed of the receptor protein itself, which is activated by binding of extracellular ATP to continuously induce the intracellular influx of cations (Na⁺, K⁺, Ca²⁺), and it further includes subtypes thereof. Specifically, the P2X receptors include P2X1 receptor, P2X2 receptor, P2X3 receptor, P2X4 receptor, P2X1 receptor, P2X6 receptor, and P2X7 receptor.

The P2X4 receptor described in accordance with the invention includes a protein with a sequence shown under Accession No. AHH33826 (human), AAA99777 (rat) or AAH05597 (mouse) in the GenBank, proteins in which amino acids at least at one position or two positions or more are substituted, homologs thereof, and partial fragments thereof. Additionally, the P2X4 receptor includes subtypes thereof and variant subtypes thereof. Further, the proteins fused with other proteins are also included within the scope of the invention, as long as the resulting proteins have the function thereof.

The gene of the P2X4 receptor according to the invention includes DNA with a sequence represented by the Accession No. AF089751 (mouse), NM_{—}011026 (mouse), XM_{—}045928 (human), NM_{—}002560 (human), or NM_{—}031594 (rat) in the GenBank, DNA in which bases at least at one position or two positions or more are substituted, complementary chains thereof (including antisense RNA), homologs thereof, and partial fragments thereof. Additionally, the gene includes the genes of the subtypes and variant types of the P2X4 receptor. Further, the DNA fused with other DNAs is also included within the scope of the invention, as long as the resulting DNA retains the function thereof.

In the present specification, the P2X4 receptor antagonist means a compound which binds to the P2X4 receptor to thereby prevent the activation of an agonist to activate the receptor. Specifically, examples thereof include low-molecular compounds, high-molecular proteins, polypeptides, polynucleotides (DNA, RNA, genes), antisense, decoy, antibodies and vaccines. These compounds may be in a form of pharmaceutically acceptable salts or in a form of prodrug. The P2X4 receptor antagonists according to with the invention are not limited to those currently known and include those possibly found newly in future.

In the present specification, the patch cramp method is a method including allowing a glass micropipette (patch electrode) to attach tightly to cell membrane at a high resistance in giga-ohms (GΩ) or more and then measuring ion currents in conditions that a very small membrane region (patch membrane) on the top opening of the electrode is electrically isolated from other regions. In particular, the inventors measured and analyzed electric currents by the whole-cell mode including opening a hole by breaking the patch membrane to record ion current flowing through the whole cell membrane except the broken patch membrane.

In the present specification, the purine skeleton of the P2X receptor antagonistic compound means a skeleton having 7H-imidazo[4,5-d]pyrimidine ring.

Additionally, the therapeutic agent of the invention may be combined and dosed with other pharmaceutical agents as a combination agent, so as (1) to supplement and/or enhance the effect of the prevention and/or treatment with the therapeutic agent of the invention, (2) to improve the pharmacokinetics and absorption of the therapeutic agent of the invention, and to reduce the dose thereof, and/or (3) to reduce adverse activities of the therapeutic agent of the invention.

The combination agent of the therapeutic agent of the invention with other pharmaceutical agents may be administrated in a blend agent containing both the components in one formulation or may be administrated in separate formulations. In the case of administrating in such separate formulations, the administration includes simultaneous administration and administration at a time interval. Additionally, the administration at a time interval may include first administration of the therapeutic agent of the invention and subsequent administration of other pharmaceutical agents; otherwise, the administration includes first administration of other pharmaceutical agents and subsequent administration of the therapeutic agent of the invention. The individual administration methods may be the same or different.

Other pharmaceutical agents described above may be low-molecular compounds or may be high-molecular proteins, polypeptides, polynucleotides (DNA, RNA, genes), antisense, decoy or antibodies or vaccines. The dose of other pharmaceutical agents may appropriately be selected, on the basis of the clinical doses thereof. Additionally, the blend ratio of the therapeutic agent of the invention and other pharmaceutical agents may appropriately be selected, taking account of the age and body weight of a subject to be administrated, the administration method, the administration time, the subject disease, the symptom, and combinations thereof. For example, other pharmaceutical agents may be used at 0.01 to 100 parts by mass with respect to one part by mass of the therapeutic agent of the invention. Appropriate two or more of other pharmaceutical agents may be combined together at an appropriate ratio, for administration. Additionally, other agents to supplement and/or enhance the effect of the prevention and/or treatment with the therapeutic agent of the invention include those possibly found in future in addition to those currently found, on the basis of the mechanism.

Diseases for which the combination agent has a prophylactic effect and/or therapeutic effect are not specifically limited. The diseases are diseases in which the effect of the prophylaxis and/or therapeutic treatment with the therapeutic agent of the invention is supplemented and/or enhanced.

Examples of other pharmaceutical agents to supplement and/or enhance the effect of the prophylaxis and/or therapeutic treatment with the therapeutic agent of the invention on respiratory diseases include antihistamine agents, anti-allergic agents such as chemical transmission substance release-suppressing agents, histamine antagonists, thromboxane synthase inhibitors, thromboxane antagonists and Th2 cytokine inhibitors; steroids; bronchodilators such as xanthine derivatives, sympathetic stimulant and parasympatholytic agent; vaccine therapeutic agents; gold formulations; Chinese herbal formulations; basic non-steroidal anti-inflammation agents; 5-lipoxygenase inhibitors; antagonists to 5-lipoxygenase-activating proteins; leukotriene synthesis inhibitors; prostaglandins; leukotriene receptor antagonists; cannabinoid-2 receptor stimulants; antitussive agents; expectorants; and extract solutions from inflammatory skin in vaccinia virus-inoculated rabbit.

Examples of antihistamine agents include diphenhydramine, diphenhydramine hydrochloride, diphenylpyraline teoclate, clemastine fumarate, dimenhydrinate, dl-chlorpheniramine maleate, d-chlorpheniramine maleate, triprolidine hydrochloride, promethazine hydrochloride, alimenazine tartrate, isothipendyl hydrochloride, homochlorcyclizine hydrochloride, hydroxyzine, cyproheptazine hydrochloride, levocabastine hydrochloride, astemizole, bepotastine, desloratadine, TAK-427, ZCR-2060, NIP-530, mometazone furoate, mizolastine, BP-294, andrast, auranofin, and acrivastine.

Among anti-allergic agents, examples of chemical transmission substance release-suppressing agents include sodium cromoglicate, tranilast, amlexanox, repirinast, ibudilast, pemirolast potassium, tazanolast, nedocromil, cromoglicate, and israpafant.

Among anti-allergic agents, examples of histamine antagonists include ketotifen fumarate, azelastine hydrochloride, oxatomide, mequitazine, terfenadine, emedastine fumarate, epinastine hydrochloride, ebastine, cetirizine hydrochloride, olopatadine hydrochloride, loratadine, and fexofenadine.

Among anti-allergic agents, examples of thromboxane synthase inhibitors include ozagrel hydrochloride and sodium imitrodast.

Among anti-allergic agents, examples of thromboxane antagonists include seratrodast, ramatroban, domitroban calcium hydrate, and KT-2-962.

Among anti-allergic agents, examples of Th2 cytokine inhibitors include suplatast tosilate.

Among steroids, examples of steroids for external use include clobetasol propionate, diflorasone acetate, fluocinonide, mometasone furoate, betamethasone dipropionate, betamethasone propionate butyrate, betamethasone valerate, difluprednate, budesonide, diflucortolone valerate, amcinonide, halcinonide, dexamethasone, dexamethasone propionate, dexamethasone valerate, dexamethasone acetate, hydrocortisone acetate, hydrocortisone butyrate, hydrocortisone propionate butyrate, deprodone propionate, prednisolone acetate valerate, fluocinolone acetonide, beclometasone propionate, triamcinolone acetonide, flumetasone pivalate, alclometasone propionate, clobetasone butyrate, prednisolone, peclometasone propionate, and fludroxycortide. Examples of Steroids for oral agents and injections include cortisone acetate, hydrocortisone, sodium hydrocortisone phosphate, sodium hydrocortisone succinate, fludrocortisone acetate, prednisolone, prednisolone acetate, prednisolone succinate, prednisolone butylacetate, sodium prednisolone phosphate, halopredone acetate, methylprednisolone, methylprednisolone acetate, sodium methylprednisolone succinate, triamcinolone, triamcinolone acetate, triamcinolone acetonide, dexamethasone, dexamethasone acetate, sodium dexamethasone phosphate, dexamethasone palmitate, paramethasone acetate and betamethasone. Examples of steroids for inhalation agents include beclometasone propionate, fluticasone propionate, budesonide, flunisolide, triancinolone, ST-126P, ciclesonide, dexamethasone palomithionate, momethasone furancarbonate, plasterone sulfonate, deflazacort, methylprednisolone suleptanate, and sodium methylprednisolone succinate.

Among bronchodilators, examples of xanthine derivatives include aminophylline, theophylline, doxophylline, cipamphylline, diprophylline, proxyphylline, and choline theophylline.

Among bronchodilators, examples of sympathetic stimulant include epinephrine, ephedrine hydrochloride, dl-methylephedrine hydrochloride, methoxyphenamine hydrochloride, isoproterenol sulfate, isoproterenol hydrochloride, orciprenaline sulfate, chlorprenaline hydrochloride, trimethoxynol hydrochloride, sulbutamol sulfate, terbutaline sulfate, hexoprenaline sulfate, tulobuterol hydrochloride, procaterol hydrochloride, fenoterol hydrobromate, formoterol fumarate, clenbuterol fumarate, mabuterol hydrochloride, salmeterol xinafoate, R,R-formoterol, tulobuterol, pilbuterol hydrochloride, ritodrine hydrochloride, bambuterol, dopexamine hydrochloride, meladrine tartrate, AR-C68397, levosalbutamol, KUR-1246, KUL-7211, AR-C89855, and S-1319.

Among bronchodilators, examples of parasympatholytic agent include ipratropium bromide, flutropium bromide, oxitropium bromide, cimetropium bromide, temiverine, thiotropium bromide, and levatropate (UK-112166).

Examples of vaccine therapy agents include paspat, asthremedin, Broncasma Berna, and CS-560.

Examples of gold formulations include sodium gold thiomalate.

Examples of basic non-steroid anti-inflammation agents include tiaramide hydrochloride, tinoridine hydrochloride, epirizole, and emorfazone.

Examples of 5-Lipoxygenase inhibitors include zyleuton, docebenon, piripost,

SCH-40120, WY-50295, E-6700, ML-3000, TMK-688, ZD-2138, dalbuferon mesylate, R-68151, E-6080, DuP-654, SC-45662, CV-6504, NE-11740, CMI-977, NC-2000, E-3040, PD-136095, CMI-392, TZI-41078, Orf-20485, IDB-18024, BF-389, A-78773, TA-270, FLM-5011, CGS-23885, A-79175 and ETH-615.

Examples of antagonists to 5-lipoxygenase-activating proteins include MK-591 and MK-886.

Examples of leukotriene synthesis inhibitors include auranofin, proglumetacin maleate, L-674636, A-81834, UPA-780, A-93178, MK-886, REV-5901A, SCH-40120, MK-591, Bay-x-1005, Bay-y-1015, DTI-0026, amlexanox, and E-6700.

Examples of prostaglandins (abbreviated as PG hereinafter) include PG receptor agonists and PG receptor antagonists.

Examples of PG receptor includes PGE receptors (EP1, EP2, EP3, EP4), PGD receptors (DP, CRTH2), PGF receptors (FP), PGI receptors (IP) and TX receptors (TP).

Examples of leukotriene receptor antagonists include pranlukast hydrate, montelukast, zaphyllukast, seratrodast, MCC-847, KCA-757, CS-615, YM-158, L-740515, CP-195494, LM-1484, RS-635, A-93178, S-36496, BIIL-284 and ONO-4057.

Examples of antitussive agents include codeine phosphate, dihydrocodeine phosphate, oxymetebanol, dextromethorphan hydrobromate, pentoxyverine citrate, dimorphan phosphate, oxeladin citrate, chloperastine, benproperine phosphate, clofedanol hydrochloride, fominoben hydrochloride, noscapine, tipepidine hibenzate, eprazinone hydrochloride, and plantago herb.

Examples of expectorants include foeniculated ammonia spirit, sodium hydrogen carbonate, potassium iodide, bromhexine hydrochloride, cherry bark extract, carbocisteine, fudostein, ambroxol hydrochloride, ambroxol hydrochloride-sustained release agent, methylcysteine hydrochloride salt, acetylcysteine, L-cysteine ethyl ester hydrochloride, and tyloxapol.

The other pharmaceutical agents are preferably steroids or sympathetic stimulant.

In the case that the therapeutic agent of the invention is to be used for the purpose described above, generally, the therapeutic agent is administrated systemically or locally in oral or parenteral forms.

The dose varies, depending on the age, the body weight, the symptoms, the therapeutic effect, the administration method, the treatment time and the like.

Generally, the therapeutic agent is orally given within a single dose range of 1 mg to 1,000 mg per adult once daily or several times daily. Otherwise, the therapeutic agent is parenterally (preferably intravenously) given once daily or several times daily, within a dose range of 1 mg to 100 mg per adult, or is given intravenously in a manner sustainable within one hour to 24 hours per day.

As described above, since the dose varies depending on the various conditions, the dose may sometimes satisfactorily be smaller than that described above or may be needed over the range.

In the case of dosing the compound for the purpose of the invention, the compound is used as an internal solid agent or an internal liquid agent for oral administration, and an injection, an external agent, a suppository, an eye drop or an inhalation agent for parenteral administration, and the like.

The internal solid agents for oral administration include tablets, pills, capsules, powders, and granules.

Examples of the capsules include hard capsule and soft capsule.

In these internal solid agents, one or more active substances are used as they are or are mixed with excipients such as lactose, mannitol, glucose, microcrystalline cellulose and starch, binders such as hydroxypropylcellulose, polyvinylpyrrolidone and magnesium metasilicate aluminate, disintegrators such as cellulose calcium glycolate, lubricants such as magnesium stearate, stabilizers, dissolution auxiliary agents such as glutamic acid and aspartic acid, and the like, and are then formulated according to general methods for use. If necessary, additionally, the resulting formulations may be coated with coating agents such as refined sugar, gelatin, hydroxypropylcellulose and hydroxypropylmethylcellulose phthalate, or may be coated with two or more layers. Further, the formulations may include capsules of absorbable substances such as gelatin.

The internal liquids for oral dosing include pharmaceutically acceptable aqueous liquids, suspensions, emulsions, syrups and elixirs. In such liquids, one or more active substances are dissolved, suspended or emulsified in diluents for general use such as distilled water, ethanol or mix solutions thereof. Further, the liquids may contain wetting agents, suspending agents, emulsifiers, sweeteners, flavor, aromatic agents, preservatives, buffers and the like.

The injections for parenteral administration include injections in solutions, suspensions, and emulsions and solid injections for use on dissolution or suspension in solvents. The injections are used by dissolving, suspending or emulsifying one or more active substances in solvents. As the solvents, for example, distilled water for injections, physiological saline, vegetable oil, alcohols such as propylene glycol, polyethylene glycol and ethanol and combinations thereof are used. Further, the injections may contain stabilizers, dissolution auxiliary agents such as glutamic acid, aspartic acid and polysorbate 80 (trade name), suspending agents, emulsifiers, soothing agents, buffers, preservatives and the like. These are prepared by sterilization at the final stage or these may be prepared by a method with aseptic procedures. Additionally, an aseptic solid agent such as a freeze-dried product may be produced and dissolved in sterilized or sterile distilled water for injections or in other solvents, before use.

Examples of the dosage form of the eye drop for parenteral administration include eye drop solutions, suspension type eye drop solutions, emulsion type eye drop solutions, eye drop solutions of dissolution type on use and eye ointment.

These eye drops may be produced by conventional methods. In the case of eye drop solutions, for example, isotonic agents such as sodium chloride and conc. glycerin, buffers such as sodium phosphate and sodium acetate, surfactants such as Polysorbate 80 (merchandise name), polyoxystearate 40 and polyoxyethylene hardened castor oil, stabilizers such as sodium citrate and sodium edetate, preservatives such as benzalkonium chloride and paraben may appropriately be selected according to the necessity for producing the eye drops. These may be produced by sterilization at the final stage or may be produced by an aseptic method.

The inhalation agents for parenteral administration include aerosol agents, powders for inhalation, or liquids for inhalation, and the inhalation agents may be in such a form that the liquids for inhalation are dissolved or suspended in water or an appropriate medium on use.

These inhalation agents are produced by conventional methods.

In the case of the liquids for inhalation, for example, preservatives such as benzalkonium chloride and paraben, colorants, buffers such as sodium phosphate and sodium acetate, isotonic agents such as sodium chloride and conc. glycerin, thickeners such as carboxyvinyl polymer, and absorption-promoting agents may appropriately be selected according to the necessity.

In the case of the powders for inhalation, lubricants such as stearic acid and salts thereof, binders such as starch and dextrin, excipients such as lactose and cellulose, colorants, preservatives such as benzalkonium chloride and paraben, absorption-promoting agents and the like may appropriately be selected according to the necessity.

In administering the liquids for inhalation, generally, sprayers such as atomizer and nebulizer are used, while in administering the powders for inhalation, generally, devices for the inhalation of pharmaceutical powder agents are used.

The other formulations for parenteral administration include external liquids, ointments, coating agents, spray agents, suppositories and pessaries for intra-vaginal administration, which contain one or more active substances and are formulated by general methods.

The spray agents may contain stabilizers such as sodium hydrogen sulfite, and buffers giving isotonicity, for example isotonic agents such as sodium chloride, sodium citrate or citric acid. The process of producing such spray agents is described in detail in U.S. Pat. Nos. 2,868,691 and 3,095,355.

### BRIEF DESCRIPTION OF THE DRAWINGS

FIG. 1 shows the ATP activity on the membrane potential of bronchial smooth muscle cells.

FIG. 2 shows the ivermectin activity on the inward current due to ATP in bronchial smooth muscle cells.

### BEST MODE FOR CARRYING OUT THE INVENTION

It is confirmed in the following Examples that the contraction of bronchial smooth muscle with ATP is a reaction through P2X4 receptor.

### EXAMPLE 1

### ATP Activity on Membrane Potential on Swine Bronchial Smooth Muscle Cells

Swine bronchial smooth muscle tissue was finely chopped and incubated at 37° C. for 40 minutes, using collagenase and papain, to isolate the cells. By applying the patch cramp method to the isolated cells, the membrane current and membrane potential on the smooth muscle cells were measured (bath (extracellular) solution: 140 mM sodium chloride, 4.7 mM potassium chloride, 1.13 mM magnesium chloride, 1.2 mM calcium chloride, 10 mM glucose, and 10 mM Hepes; pipette (intracellular) solution: 140 mM potassium chloride, 1.13 mM magnesium chloride, 10 mM glucose, 10 mM Hepes, 0.5 mM ethyl glycol-bis(β-aminoethyl ether)-N,N,N′,N′-tetraacetic acid; the individual solutions of the aforementioned compositions were adjusted to pH 7.2, using sodium hydroxide (for the extracellular solution) or potassium hydroxide (for the intracellular solution), for use.

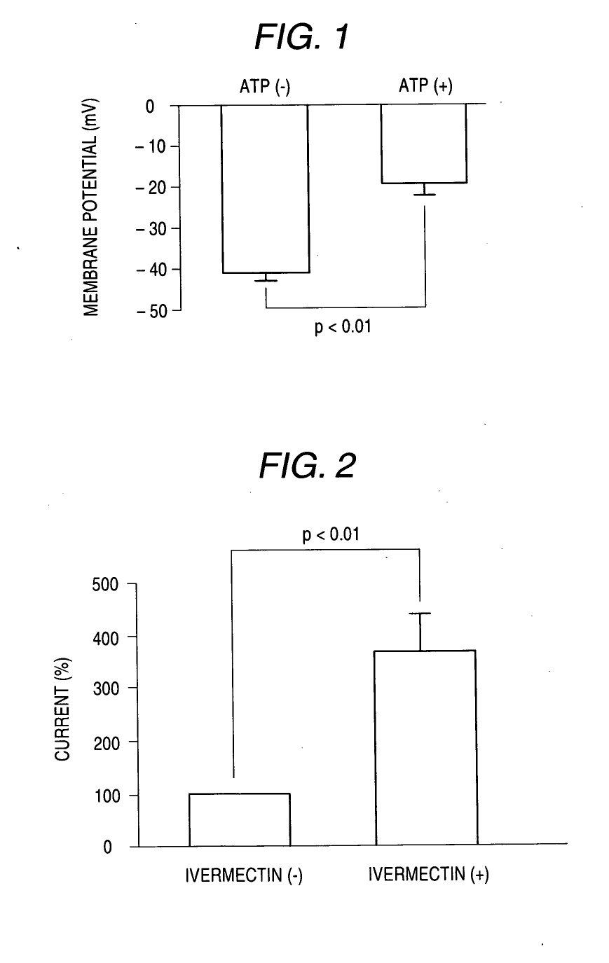

When ATP was given while the membrane potential was maintained at −40 mV, inward current passed. When the electric current was fixed, additionally, the cellular membrane potential was depolarized from −40 mV to −20 mV, due to ATP. The results are shown in FIG. 1.

It is known that phospholipase C (PLC) is activated when P2Y receptor is involved as a purine receptor. After treatment with U-73122 (100 μM) as a PLC suppressing agent and subsequent ATP dosing, no suppression of the current occurred.

Therefore, it is suggested that the current is not via the P2Y receptor but via the P2X receptor.

It has been known so far that the increase of intracellular calcium concentration plays an important role for the contraction of smooth muscle. Thus, examination was done so as to elucidate whether or not the inward current shown in FIG. 1 was involved in calcium influx. When SKF 96365 suppressing the receptor-agonistic calcium influx and potential-dependent calcium channel was treated, the inward current was suppressed by about 50%. When verapamil suppressing only the potential-dependent calcium channel was treated, alternatively, the current was never suppressed. This indicates that the inward current due to ATP (about 50%) contains calcium and is not the potential-dependent calcium channel but an influx via the P2X receptor.

The results mentioned above suggest that the membrane potential of bronchial smooth muscle cells is depolarized through the P2X receptor via ATP and that calcium is involved in the reaction.

### EXAMPLE 2

### Activities of P2X Receptor Antagonist and P2X4 Enhancer on Inward Current Via ATP on Swine Bronchial Smooth Muscle Cells

By the same method as in Example 1, the following experiment was done so as to elucidate which subtype of the P2X receptor was involved.

After the termination of ATP administration, the inward current via ATP in swine bronchial smooth muscle cells gradually resumed the baseline value.

A number of antagonists to P2X receptor have been known. Currently, not any specific antagonist to a finely classified subtype of the P2X receptor has been developed. Therefore, typical antagonists pyridoxal phosphate-6-azophenyl-2′,4′-disulfonic acid (PPADS) and suramin were treated. No suppression of the inward current occurred. The P2X4 receptor and the P2X6 receptor are known to be insensitive to both the PPADS and suramin.

When ivermectin as a P2X4 receptor enhancer was treated, then, the current was enhanced to 3.5-fold as shown in FIG. 2.

The results indicate that the inward current via ATP in bronchial smooth muscle cells is via the P2X4 receptor.

The fact that ATP contracts airway smooth muscle via calcium influx and the results in Examples 1 and 2 demonstrate that ATP caused intracellular calcium influx via the P2X4 receptor to depolarize the membrane potential. In other words, antagonists to the P2X4 receptor existing in bronchial smooth muscle can be used for therapeutically treating respiratory diseases caused by bronchocontraction such as asthma.

### INDUSTRIAL APPLICABILITY

Since the therapeutic agent for respiratory diseases according to the invention is antagonistic to the P2X4 receptor in bronchial smooth muscle to suppress the contraction of bronchial smooth muscle, the therapeutic agent is useful for the prevention and/or treatment of respiratory diseases such as asthma and chronic obstructive lung diseases.

1. A method for treating respiratory diseases, comprising administering to a subject an effective amount of a compound antagonistic to a P2X receptor.

2. The method according to claim 1, wherein the compound antagonistic to a P2X receptor is a compound satisfying the following conditions:
(1) the compound suppressing an inward current induced by ATP in the measurement with the patch cramp method;
(2) the compound antagonistic to a receptor which is not antagonized with pyridoxal phosphate-6-azophenyl-2′,4′-disulfonic acid and/or suramin; and
(3) the compound suppressing an inward current enhanced by ivermectin in the measurement with the patch cramp method.

3. The method according to claim 1, wherein the compound antagonistic to a P2X receptor has a purine skeleton.

4. The method according to claim 1, wherein the respiratory disease is asthma.

5. The method according to claim 1, wherein a bronchus is dilated.

6. (canceled)

7. The method according to claim 4, wherein the P2X receptor is a P2X4 receptor.

8. The method according to claim 5, wherein the P2X receptor is a P2X4 receptor.

9. A method for dilating a bronchus, comprising administering to a subject an effective amount of a compound antagonistic to a P2X receptor
